(19) United States
(12) Patent Application Publication
(10) Pub. No.: US 2007/0026455 A1
(43) Pub. Date: Feb. 1, 2007

(54) Method of producing hybridoma and utilization thereof

(75) Inventors: Masahiro Tomita, Tsu-shi (JP); Shigeru Fujimoto, Osaka-shi (JP)

Correspondence Address: EDWARDS & ANGELL, LLP, P.O. BOX 55874, BOSTON, MA, 02205, US

(73) Assignees: Mie TLO Co., Ltd., Mie (JP); Japan EnviroChemicals, Ltd., Osaka-shi (JP)

(21) Appl. No.: 10/547,810

(22) PCT Filed: Mar. 5, 2004
(86) PCT No.: PCT/JP04/02926

§ 371 Date: May 30, 2006

(30) Foreign Application Priority Data
Mar. 6, 2003 (JP) 2003-059667

(51) Int. Cl. G01N 33/53; C07H 21/04; C12P 21/04; C12P 21/08; C07K 16/18

(52) U.S. Cl. 435/7.1; 435/70.21; 435/451; 530/388.1; 536/23.53

(57) ABSTRACT

The present invention provides a method of producing an antibody-producing hybridoma, which includes fusing a B cell immunized in vitro and a myeloma cell, and which is more efficient and practical than the conventional methods, more specifically a method of producing an antibody-producing hybridoma, which includes fusing a B cell immunized with an antigenic substance in the coexistence of a cytokine and a glycolipid in vitro, and a myeloma cell, and use thereof.

### FIELD OF THE INVENTION

The present invention relates to a method of producing an antibody-producing hybridoma and use thereof.

### BACKGROUND ART

In recent years, environmental pollution with endocrine disruptors (also referred to as “environmental hormones”) has been a problem, and there is need to measure and analyze endocrine disruptors and degradation products thereof present in the environment, and to urgently utilize the results for environmental conservation. Conventionally, various methods of measuring and analyzing endocrine disruptors have been known, and a method using a gas chromatography-mass spectrometer (GC-MS) is currently the mainstream. However, this method has some drawbacks, including inability to obtain necessary sensitivity to quantify endocrine disruptors, which occur in very small amounts, necessity for high-rate concentration by solvent extraction and the like to achieve quantitation, use of very expensive equipment requiring high skills for operation, long time required per sample to perform concentration, extraction and detection (several weeks for some substances such as dioxins) and the like.

As a method of detecting endocrine disruptors quickly with high sensitivity from a viewpoint totally different than that of conventional methods, which can resolve these problems, a method using a monoclonal antibody has been proposed. Monoclonal antibodies are antibodies that specifically recognize only one particular epitope out of some epitopes (antigen determinants) present in an antigenic substance, and are used widely in a number of fields for their very high specificity, including identification of structurally similar substances, detection of trace components, and one-step purification of desired antigenic substances.

A currently widely used method of preparing a monoclonal antibody comprises fusing a B cell such as a splenocyte or lymph node cell previously immunized with a desired antigenic substance (an immunized B cell is hereinafter also referred to as “immunized cell”) and an immortal B-cell-derived myeloma cell to yield an antibody-producing cell (hybridoma). According to this method, it is possible to semipermanently proliferate a hybridoma that produces a particular monoclonal antibody to semipermanently produce the monoclonal antibody.

Immunization for preparation of the above-described immunized cell has been conventionally often conducted by an in vivo immunization method. When using a biologically toxic substance like an endocrine disruptor as the antigenic substance, however, it is desirable that immunization be conducted in vitro (see, for example, Boss et al., Methods Enzymol. 121, 27-33 (1986)). In vitro immunization offers advantages, including a trace amount of antigen required (immunization possible with as small as 1 μg to 10 μg of antigen), a short immunization time required (immunization completed in a very short period of 3 to 5 days) and the like. From the viewpoint of establishment of a more efficient and practical method of monoclonal antibody production, therefore, it is desirable to fuse a cell immunized in vitro to yield a desired antibody-producing hybridoma.

### DISCLOSURE OF THE INVENTION

However, the incidence of hybridoma obtained by cell fusion of a B cell immunized in vitro is about 10% to 30%, which is lower than that by conventional in vivo immunization (hybridoma incidence by in vivo immunization: about 40% to 50%), and there is a demand for development of a more efficient method of hybridoma production. The above-described hybridoma obtained by cell fusion of a B cell immunized in vitro has another drawback of poor practical applicability because many of the monoclonal antibodies produced thereby are of the IgM type, which is a structurally unstable pentamer.

The present invention has been made with the aim of resolving the above problems, and is directed to providing a more efficient and practical method of producing an antibody-producing hybridoma, which comprises selecting a B cell immunized in vitro using an immune antigen, and fusing it with a myeloma cell.

To resolve the above problems, the present inventors tried immunization of a splenocyte in vitro in the coexistence of interleukin-4 and lipopolysaccharide, which were added for the purpose of an antibody class switch from the IgM type to the IgG type. For the immunization, a derivative (hapten) of di-2-ethylhexyl phthalate (DEHP) was used as the antigenic substance. Although DEHP is widely and commonly used as a plasticizer, it was included in the eight substances to be preferentially subjected to a risk assessment out of substances, for which evidence of at least one endocrine disrupting action on organisms had been given, in the report (draft) on a priority list of endocrine disruptors, which was prepared by the European Commission (EU) in June 2000; as such, DEHP is an endocrine disrupter against which immediate actions are particularly needed.

The present inventors examined monoclonal antibodies produced by a hybridoma prepared by fusing an immunized cell obtained by the above-described attempt with a myeloma cell using the pulsed electric field (PEF) method, and confirmed a class switch to the IgG type; separately, the present inventors unexpectedly found that the incidence of hybridomas obtained by this technique was as high as nearly 50%, and completed the present invention based on such findings. Accordingly, the present invention provides the following:

(1) A method of producing an antibody-producing hybridoma, which comprises fusing a B cell, previously immunized with an antigenic substance in the coexistence of a cytokine at a sufficient concentration to selectively produce an IgG type antibody, and a glycolipid in vitro, and a myeloma cell.

(2) The method described in (1) above, wherein the cytokine concentration ranges from 1 ng/ml to 100 ng/ml.

(3) The method described in (1) above, wherein the cytokine concentration ranges from 5 ng/ml to 30 ng/ml.

(4) The method described in any of (1) to (3) above, wherein a cell is immunized in the absence of Phytolacca americana lectin and/or killed Staphylococcus aureus cells coated with protein A.

(5) The method described in any of (1) to (4) above, wherein the cytokine is interleukin-4 and the glycolipid is lipopolysaccharide.

(6) The method described in any of (1) to (5) above, wherein the antigenic substance is an endocrine disruptor or a protein containing a peptide.

(7) The method described in any of (1) to (6) above, which comprises cell fusion by the pulsed electric field method.

(8) The method described in (6) above, which comprises cell fusion using (a) and (b) below:

(a) an immunized cell joined to a complex comprising both the antigen used for immunization and one member of a pair of specific couplings, and

(b) a myeloma cell joined to the other member of the pair of specific couplings.

(9) A monoclonal antibody produced by a hybridoma prepared by the method described in any of (1) to (8) above.

(10) A cell fusion reagent containing at least both a cytokine at a sufficient concentration to selectively produce an IgG type antibody, and a glycolipid.

(11) The cell fusion reagent described in (10) above, wherein the cytokine is interleukin-4, and the glycolipid is lipopolysaccharide.

(12) A method of monoclonal antibody production, which comprises a step using an antibody-producing hybridoma obtained by fusing a B cell, previously immunized with an antigenic substance in the coexistence of a cytokine at a sufficient concentration to selectively produce an IgG type antibody, and a glycolipid in vitro, and a myeloma cell.

(13) The production method described in (12) above, wherein the antigenic substance is an endocrine disruptor or a protein containing a peptide.

(14) A method of screening for an antigenic substance, which comprises a step using a monoclonal antibody produced by an antibody-producing hybridoma obtained by fusing a B cell, previously immunized with an antigenic substance in the coexistence of a cytokine at a sufficient concentration to selectively produce an IgG type antibody, and a glycolipid in vitro, and a myeloma cell.

(15) The screening method described in (14) above, wherein the antigenic substance is an endocrine disruptor or a protein containing a peptide.

(16) A method of hybridoma production, which comprises cell fusion of (a) and (b) below by the pulsed electric field method:

(a) an immunized cell joined to a complex comprising both the antigen used for immunization and one member of a pair of specific couplings, and

(b) a myeloma cell joined to the other member of the pair of specific couplings.

### BRIEF DESCRIPTION OF THE DRAWINGS

FIG. 1 is a photomicrograph showing the results of a visualized analysis using a specific fluorescent label of immunized cells of Example 1 before cell fusion following in vitro immunization.

FIG. 2 is a photomicrograph showing the results of a visualized analysis using a specific fluorescent label of immunized cells of Comparative Example 1 before cell fusion following in vitro immunization.

### DETAILED DESCRIPTION OF THE INVENTION

The present invention is hereinafter described in detail.

The method of the present invention of producing an antibody-producing hybridoma comprises two largely divided steps: [1] a step of immunizing a B cell with an antigenic substance in vitro in the coexistence of a cytokine and a glycolipid, and [2] a step of selecting the above-described immunized B cell (immunized cell) with an immune antigen, then fusing the cell with a myeloma cell to yield a hybridoma.

The term “immunizing a B cell with an antigenic substance in the coexistence of a cytokine and a glycolipid” as used herein means that immunization is conducted in a way that allows a cytokine and a glycolipid to sufficiently associate with B cells and other immunocompetent cells; provided that immunization takes place in this way, the order of addition and quantities of the B cell, antigenic substance, cytokines, and glycolipid are not subject to limitation.

In step [1] above of the present invention, it is necessary that a B cell be immunized with an antigenic substance in the coexistence of both a cytokine and a glycolipid; the effect of the present invention described below cannot be achieved in the sole presence of either member of the combination. This is because of a drawback that B cell activation is insufficient in the presence of a cytokine alone during B cell immunization in vitro, and also because of a drawback that a class switch from IgM to IgG does not occur in the presence of a glycolipid alone. Additionally, even if both a cytokine and a glycolipid are coexistence with a B cell at a time other than during B cell immunization with an antigenic substance, cell fusion of the B cell with a myeloma cell could not achieve a high incidence of antibody-producing hybridomas.

The cytokine used in the present invention is not subject to limitation, as long as it is a publicly known cytokine that serves as a signal transducer in the immune system, and includes, for example, not only interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-9 (IL-9), interleukin-10 (IL-10), interleukin-11 (IL-11), interleukin-12 (IL-12), interleukin-13 (IL-13), interleukin-14 (IL-14), interleukin-15 (IL-15), interleukin-16 (IL-16), interleukin-17 (IL-17), interleukin-18 (IL-18) and the like, but also interferon (IFN), tumor necrosis factor (TNF), colony-stimulating factor (CSF), erythropoietin (EPO) and the like. Particularly preferred is IL-4 because it is capable of inducing a class switch from IgM to IgG. Although the above-described interleukins may be derived from any of mouse, human, and rat, it is preferable that an interleukin of mouse origin be used because the mouse is a preferred subject of immunization. Commercially available cytokines can be used appropriately.

While the amount of cytokine added to be coexistent during immunization is not subject to limitation, it is preferable, from the viewpoint of optimum concentration in cell culture, that IL-4, for example, be added to obtain a final concentration of 0.1 ng/ml to 100 ng/ml, more preferably a final concentration of 5 ng/ml to 30 ng/ml.

In another aspect regarding the amount of cytokine added to be coexistent during immunization, wherein the cytokine is added to obtain a final concentration exceeding 1 ng/ml, and enabling more selective acquisition of B cells that produce an IgG type antibody, it is preferable, from the viewpoint of optimum concentration in cell culture, that the cytokine be added to obtain a final concentration of 1 ng/ml to 100 ng/ml, more preferably a final concentration of 5 ng/ml to 30 ng/ml.

In the present invention, the effect of the present invention described below can be achieved even when using two kinds or more of the cytokines shown for exemplification above. In this case, it is preferable that the total amount of the two kinds or more of cytokines used be within the range described above.

Any glycolipid can be used for the present invention, as long as it is a substance containing both a saccharide and a lipid (both a water-soluble sugar chain and a fat-soluble group) in the molecular structure thereof. For example, lipopolysaccharides (derived from Escherichia coli, Salmonella enteritidis, Salmonella typhimurium, Serratia marcescens and the like) (e.g., various forms of lipopolysaccharides obtained using various extraction methods and artificial lipopolysaccharides described in SIGMA General Catalogue 2000-2001 Japanese version, LIPOPOLYSACCHARIDES) can be mentioned. Commercially available glycolipids can be used without limitation.

Although the concentration of the glycolipid added to be coexistent during immunization is not subject to limitation, it is preferable that a lipopolysaccharide, for example, be added to obtain a final concentration of 5 μg/ml to 50 μg/ml, more preferably a final concentration of 10 μg/ml to 20 μg/ml.

In the present invention, the effect of the present invention described below can be achieved even when using two kinds or more of the glycolipids shown for exemplification above. In this case, it is preferable that the total amount of the two kinds or more of glycolipids used be within the range described above.

A B cell used for in vitro immunization in the present invention can, for example, be obtained as a splenocyte or lymph node cell by extirpating the spleen or a lymph node aseptically by a conventional method from an animal conventionally used for in vivo immunization, such as mouse, goat, sheep, rabbit, rat, guinea pig, or chicken, washing, and disrupting the spleen or lymph node. Because of its abundance in B cells and other immunocompetent cells, splenocytes are preferably used as B cells. B cells are prepared by suspending cells, in an appropriate antibiotic-containing suspending medium (when using splenocytes, for example, they are suspended in a kanamycin-containing RPMI1640 (Roswell Park Memorial Institute 1640) (manufactured by Nissui Pharmaceutical Co., Ltd.) and the like) so that they can be used for in vitro immunization.

The myeloma cell used in the present invention is not subject to limitation; for example, conventionally known NS-1, P3U1, Sp2/O, PAI and the like can be mentioned, with preference given to PAI because of high fusion efficiency. A myeloma cell previously subcultured according to a method in common use in the art may be used.

Although the order of addition of B cell, antigenic substance, cytokine, and glycolipid in step [1] of the present invention is not subject to limitation, it is preferable that these be added in the order of B cell, antigenic substance, cytokine, and glycolipid to increase the efficiency of B cell sensitization to the desired antigenic substance.

The in vitro B cell immunization in step [1] above can be conducted appropriately in accordance with traditionally known Boss' method (Boss: Methods Enzymol. 121, 27-33 (1986)). When using splenocytes, for example, B cell immunization is conducted per procedures (1) to (3) below.

(1) A suspension containing splenocytes prepared as described above is allowed to stand with the addition of an appropriate amount of each antigen solution previously prepared as described below.

(2) After the suspension is allowed to stand, a complete medium containing 40% FCS (fetal calf serum) (60% RPMI1640+100 μg/ml kanamycin sulfate+2 mM L-glutamine+50 μM β-mercaptoethanol+40% FCS) is added. The addition of a cytokine and a glycolipid, which is a feature of the present invention, is, for example, performed after this addition of the complete medium containing 40% FCS. Subsequently, cultivation is conducted in a 5% carbon dioxide incubator for about 3 to 5 days.

(3) After cultivation, the culture broth is centrifuged, the resulting precipitate is suspended in an antibiotic-containing RPMI1640, the resulting suspension is centrifuged, and the resulting precipitate is washed and then suspended in the antibiotic-containing RPMI1640 to yield a suspension of the immunized cell immunized in vitro.

In procedure (1) above, it is preferable, from the viewpoint of the ability to increase immunization efficiency, that an adjuvant be further added. As the adjuvant, a conventionally known one may be chosen as appropriate without limitation. The adjuvant used may be a commercial product; for example, N-acetylmuramyl-L-alanyl-D-isoglutamine (manufactured by SIGMA), Freund's complete adjuvant (manufactured by Difco Laboratories), Freund's incomplete adjuvant (manufactured by Difco Laboratories), RIBI adjuvant (manufactured by Corixa Corporation) and the like can be mentioned, with preference given to N-acetylmuramyl-L-alanyl-D-isoglutamine for the purpose of efficient immunization in vitro. Although the amount of adjuvant added is not subject to limitation, it is preferable, from the viewpoint of improvement in immunization efficiency, that the adjuvant be added at 5 μg/ml to 50 μg/ml, more preferably 20 μg/ml to 40 μg/ml, relative to the antigenic substance.

As the method of cell fusion used in step [2] of the present invention, conventionally known methods such as the method using Sendai virus, the method using polyethylene glycol (PEG) as a fusogen, the pulsed electric field (PEF) method, and laser radiation can be used without limitation. In particular, the cell fusion method using PEG is generally widely used because it is relatively easy to perform, but the aldehydes resulting from PEG oxidation are known to be potently cytotoxic and to affect hybridoma growth. This method is also faulty in that a great deal of labor and time is taken to obtain the desired monoclonal antibody because of nonspecific cell fusion occurring not only between an immunized cell and a myeloma cell but also between immunized cells and/or between myeloma cells. Therefore, in the present invention, it is preferable that cell fusion be conducted using the PEF method, out of the above-described methods of cell fusion, because it enables selective cell fusion of an immunized cell and a myeloma cell without using a cytotoxic substance, and also enables preparation of a hybridoma capable of producing the desired antigen-specific monoclonal antibody at an efficiency 10 to 20 times as high as that obtained using the above-described method of cell fusion using PEG.

The PEF method is a method of cell fusion comprising preparing an immunized cell-antigenic substance-avidin-biotin-myeloma cell complex using biotin/avidin crosslinkage based on the strong affinity of biotin and avidin from a combination of a first complex comprising an immunized cell and avidin bound thereto via an antigenic substance and a second complex comprising a myeloma cell and biotin bound thereto, and applying a high-voltage square pulse thereto. The biggest feature of this PEF method resides in that an immunized cell alone can be selectively used for the generation of a first complex using an avidin-bound antigenic substance because the immunized cell can be selected in advance using an antigen via an antigen receptor on the cell (Lo, M. M. S. et al.: Nature 310, 792-794 (1984); Masahiro Tomita et al.: Biochem. Biophy. Acta. 1055, 199-206 (1990); Masahiro Tomita et al.: J. Immunol. Methods. 251, 31-43 (2001); Tsong, T. Y et al.: Methods Enzymol. 220, 238-246 (1993); Masahiro Tomita et al.: Protein, Nucleic Acid and Enzyme 45, 600-606 (2000)).

Specifically, cell fusion may be conducted per conventionally known procedures as described below. The following describes an example case in which the derivative (hapten) of di-2-ethylhexyl phthalate (DEHP) described below is used as the antigenic substance in combination with a splenocyte as the B cell. In the case of other antigenic substances, cell fusion can be conducted in the same manner.

(1) N-hydroxysuccinimide and 1-ethyl-3-(3-dimethylaminopropyl)carbodiimide hydrochloride are added to a previously prepared DEHP derivative (hapten) containing a carboxyl group in an appropriate solvent (DMSO and the like) to esterify the derivative. Avidin is added to the resulting ester to yield a conjugate of the derivative (hapten) and the avidin bound thereto.

(2) The above-described conjugate is mixed in a suspension of immunized cells, the suspension is gently rotated and centrifuged, the resulting precipitate is washed with an appropriate antibiotic-containing suspending medium (for example, the above-described kanamycin-containing RPMI1640) and suspended in the above-described suspending medium. Thus, a first complex comprising an immunized cell and avidin bound thereto via the antigenic substance is generated.

(3) Myeloma cells, previously subcultured by a conventional method, are washed and suspended in an appropriate suspending medium (for example, PBS) to yield a suspension containing myeloma cells; separately, NHS-biotin (N-hydroxysuccinimide-biotin) is suspended in an appropriate solvent (for example, DMF) to yield a biotin-containing suspension. These ingredients are mixed, rotated at 37° C. in a 5% carbon dioxide incubator, and centrifuged, and the resulting precipitate is washed with the above-described suspending medium (kanamycin-containing RPMI1640) and suspended in the suspending medium. Thus, a second complex comprising a myeloma cell and biotin bound thereto is generated.

(4) A suspension containing the first complex prepared in (2) above and a suspension containing the second complex prepared in (3) above are mixed. The mixing ratio is preferably such that the ratio of splenocyte suspension containing immunized cells and myeloma cells is 10:1 to 1:2, more preferably 1:1. The mixed suspension is centrifuged, and the resulting precipitate is suspended in the above-described suspending medium. The suspension is centrifuged, allowed to stand for an appropriate time, and rotated. After rotation, centrifugation is conducted, and the resulting precipitate is suspended in an isotonic sucrose buffer (0.25 M sucrose+2 mM sodium dihydrogen phosphate/disodium hydrogen phosphate (pH 7.2)+0.1 mM magnesium chloride+0.1 mM calcium chloride). Thus, an immunized cell-DEHP derivative (hapten)-avidin-biotin-myeloma cell complex based on the strong affinity of biotin and avidin is generated.

(5) A suspension containing the immunized cell-DEHP derivative (hapten)-avidin-biotin-myeloma cell complex obtained in (4) above is added to platinum plates of the preparation type at 1 ml/plate, and electric pulses are applied. This electric pulse application is achieved by applying direct-current high-voltage square pulses using, for example, a commercially available cell fusion apparatus such as the electro square porator T820 (manufactured by BTX), ECM830 (manufactured by BTX), or ECM2001 (manufactured by BTX). Pulse application conditions are usually 2 kV/cm (10 μsec×4 times) or 3 kV/cm (10 μsec×4 times), with preference given to 2 kV/c=(10 μsec×4 times) because of prevention of cell damage. Thus, immunized cells and myeloma cells, which are crosslinked, are fused selectively to yield a hybridoma.

The hybridoma prepared by the method of the present invention is cultured by a conventional method in preparation for screening.

After addition of RPMI complete medium (90% RPMI1640+10% FCS+100 μg/ml kanamycin sulfate+2 mM L-glutamine+50 μM β-mercaptoethanol), for example, the above-described hybridoma is cultured. This cultivation is normally carried out on a 96-well microplate or a 48-well microplate; for example, as is conventionally known, the supernatant is exchanged (for example, 0.1 ml/well portions) with a HAT-containing RPMI complete medium (containing 100 μM hypoxanthine+0.4 μM aminopterin+16 μM thymidine) for 1.5 to 2 weeks, the supernatant is then exchanged (for example, 0.1 ml/well portions exchanged) with a HT-containing RPMI complete medium (containing 100 μM hypoxanthine+16 μM thymidine) for 1.5 to 2 weeks, and this is followed by medium exchanges with RPMI1640 complete medium.

Although various methods can be used to screen for a desired hybridoma, this screening can be conducted by, for example, the ELISA (enzyme-linked immunosorbent assay) method. Hybridomas testing positive for antibody activity in the ELISA method may be cloned using a technique in common use in the art, for example, limiting dilution. It is possible to determine the antibody titer of the supernatant of a cloned hybridoma by the above-described method, and to select a hybridoma that produces a high-titer antibody stably, so as to obtain a desired monoclonal antibody-producing hybridoma.

Production and purification of a monoclonal antibody using a hybridoma can be conducted using a method known per se. Examples of methods of antibody production and purification are given on pages 46-71 and 85-110 in “Enzyme Immunoassay” for example; salting-out (Na₂SO₄, (NH₄)₂SO₄), ion exchangers (DEAE, QAE, CM/cellulose, Sephadex, Sepharose, Servacel and the like), hydrophobicity chromatography (L-phenylalanyl-Sepharose and the like), gel filtration (Sephadex G-200, Bio-Gel p-300 and the like), electrophoresis (zone electrophoresis using agarose gel), isoelectric focusing, isotachophoresis and the like), ultracentrifugation (sucrose density gradient centrifugation), affinity chromatography (immobilized protein A (Protein-A Sepharose, Protein-A superose and the like)) and the like can be mentioned.

Furthermore, step [2] comprises cell fusion of (a) and (b) below by the pulsed electric field method: (a) an immunized cell joined to a complex containing both the antigen used for immunization and one member of a pair of specific couplings, and (b) a myeloma cell joined to the other member of the pair of specific couplings. The term “pair of specific couplings” refers to a particular combination of two compounds possessing specific affinity for each other; for example, avidin-biotin, streptavidin-biotin, antigen (different from that used for immunization)-antibody, receptor-hormone, receptor-ligand, receptor-agonist, receptor-antagonist, enzyme-substrate, lectin-carbohydrate, nucleic acid (RNA or DNA)-hybridizing sequence, Fc receptor or mouse IgG-protein A, mouse IgG-protein G and the like can be mentioned. A pair of specific couplings can be joined to an immunized cell and a myeloma cell directly or indirectly via a linker or the like. This step makes it possible to increase the incidence of hybridomas more efficiently.

The present invention is a method of hybridoma production that follows steps [1] and [2] described above.

According to the production method of the present invention, wherein a B cell is immunized in the coexistence of a cytokine and a glycolipid, the hybridoma prepared produces a IgG type monoclonal antibody, which is a monomer and structurally stable, at a much higher ratio than of the IgM type, compared to B cell immunization conducted in the same manner as the present invention but in the absence of the cytokine and glycolipid (Comparative Examples 1 and 2 described below).

Thus, the present inventors found that even when a B cell is immunized in vitro and cell fusion is conducted using this immunized B cell, an IgG type monoclonal antibody can be produced efficiently. The class of the above-described monoclonal antibody can be determined by the sandwich ELISA method (Tandem method), which is a publicly known method.

Furthermore, the method of the present invention allows a hybridoma to emerge at a much higher rate (hybridoma positivity rate) after cell fusion in the coexistence of a cytokine and a glycolipid during immunization, compared to immunization conducted in the same manner as the present invention but in the absence of the cytokine and the glycolipid (Comparative Examples 1 and 2 described below). Furthermore, hybridoma positivity rates of 40% to 85% can be achieved by conducting cell fusion by the PEF method (these hybridoma positivity rates are 2 to 40 times as high as those obtained with immunization conducted in the same manner as the present invention but in the absence of the cytokine and glycolipid).

As such, the present invention realizes a hybridoma production method that is much more efficient and practical than the conventional method.

As the antigenic substance used in the method of the present invention, an appropriate conventionally known substance or a substance that will be newly obtained in the future can be used without limitation. In particular, by using one of endocrine disruptors (environmental hormones), against which an immediate response has been recently called for, as the antigenic substance, it is possible to produce a hybridoma capable of producing a monoclonal antibody that can specifically bind to the endocrine disruptor more efficiently and practically than the conventional method. The term “endocrine disruptor” as used herein refers to an exogenous agent that interferes with the various processes, such as biosynthesis, storage, secretion, in vivo transport, binding, action or elimination, of various hormones in the body, which are responsible for homeostasis, reproduction, development or behavior, and is herein understood to encompass all substances that are generally recognized as endocrine disruptors. As examples of such endocrine disruptors, female hormones, male hormones, thyroid hormones, alkylphenol ethoxylates, alkylphenols, resin components, resin plasticizers, chlorophenols, and other substances can be mentioned.

As examples of the female hormones, estrogen, estradiol (E2), estrone (E1), estriol (E3) and the like can be mentioned. As examples of male hormones, androgen, testosterone, dehydroepiandrosterone, androstenedione and the like can be mentioned. As examples of the thyroid hormones, thyroxine (T3) triiodotyronine (T4) and the like can be mentioned, including in vivo metabolites thereof in the form of conjugates (for example, glucuronides, sulfate conjugates and the like).

As examples of the alkylphenol ethoxylates (APEs), NPEs (nonylphenol ethoxylates, for example, NP2EO (average ethylene oxide chain number: 2), NP5EO (average ethylene oxide chain number: 5), NP10EO (average ethylene oxide chain number: 10), NP10EC (average ethylene oxide chain number: 10, terminal OH→carboxylic acid)), OPE (octylphenol ethoxylate) and the like can be mentioned.

As examples of the alkylphenols (APs), DP (4-dodecylphenol), EP (4-ethylphenol), HP (heptylphenol), IPP (4-isopentylphenol), 2-OP (2-octylphenol), 4-NP (4-nonylphenol), 4-OP (4-octylphenol), 4-sBP (4-sec-butylphenol), 4-tBP (4-t-butylphenol), 4-tPP (4-t-pentylphenol), 4-tOP (4-t-octylphenol) and the like can be mentioned.

As examples of the resin components (PRCs), BPA (bisphenol A), 4,4′-EBP (4,4′-ethylidenebisphenol), BHPM (bis(p-hydroxyphenyl)methane), 2,2′-BHPP (2,2′-bis(4-hydroxyphenyl)-1-propanol), 2,2′-BMHPP (2,2′-bis(m-methyl-p-hydroxyphenyl)propane), 4,4′-BHPP (4,4′-bis(p-hydroxyphenyl)pentanoic acid), 4,4′-DDE (4,4′-dihydroxydiphenyl ether), 4,4′-DOHDS (4,4′-dihydroxydiphenyl sulfone), 4,4′-DClDS (4,4′-dichlorodiphenyl sulfone), BHEDBrPS (bis[4-(2-hydroxyethoxy)-3,5-dibromophenyl]sulfone), BHEPS (bis[4-(2-hydroxyethoxy)phenyl]sulfone), 4,4′-DDE (4,4′-dihydroxydiphenyl ether), p,p′-DBP (p,p′-dihydroxybenzophenone), and HBP (4-hydroxybiphenol and the like) can be mentioned.

As the resin plasticizers (PPs), BBP (butylbenzyl phthalate), DBP (dibutyl phthalate), DCHP (dicyclohexyl phthalate), DEP (diethyl phthalate), DEHP (di-2-ethylhexyl phthalate), DEHA (diethylhexyl adipate), DHP (dihexyl phthalate), DPP (di-n-pentyl phthalate), DPrP (dipropyl phthalate) and the like can be mentioned.

As the chlorophenols (CPs), 2-CP (2-chlorophenol), 3-CP (3-chlorophenol), 4-CP (4-chlorophenol), 2,3-CP (2,3-dichlorophenol), 2,4-CP (2,4-dichlorophenol), 2,5-CP (2,5-dichlorophenol), 2,6-CP (2,6-dichlorophenol), 2,3,4-CP (2,3,4-trichlorophenol), 2,4,5-CP (2,4,5-trichlorophenol), 2,4,6-CP (2,4,6-trichlorophenol), 2,3,4,5-CP (2,3,4,5-tetrachlorophenol), 2,3,4,6-CP (2,3,4,6-tetrachlorophenol), PCP (pentachlorophenol) and the like can be mentioned.

As other endocrine disruptors, polychlorodibenzo-p-dioxins (PCDDs), represented by 2,3,7,8-tetrachlorodibenzo-p-dioxin (TCDD), polychlorodibenzofurans (PCDFs), represented by 2,3,7,8-tetrachlorodibenzofuran (TCDF), polychlorobiphenyl (PCB), benzophenone, benzopyrane, chlorobenzene, bromonaphthol, nitrotoluene, tributyl tin, various agricultural chemicals, heavy metals (for example, Cd, Hg, Pb and the like), synthetic estrogens (centchroman, ethynylestradiol, diethylstilbestrol, hexestrol, 2-hydroxyestradiol, tamoxifen, raloxifene and the like), foods, and food additives (for example, BHA (butylhydroxyanisole), equol, enterolactone, phytoestrogen, coumestrol, formononetin, daidzein, biochanin A, genistein and the like) can be mentioned.

The method of the present invention can also be embodied appropriately using a protein as the antigenic substance. The protein is understood to encompass peptides, for example, human insulin.

When using a low-molecular compound having relatively low antigenicity, for example, DEHP, as the antigenic substance in the production method of the present invention, it is recommended that a complex comprising the antigenic substance and a carrier protein bound thereto be used for the purpose of increasing the antigenicity thereof. The complex can be prepared by, for example, binding a substance represented by the formula below:
A-R
(wherein R represents COOH, NH₂ or SH, and A represents a group that becomes an antigenic substance (preferably the above-described endocrine disrupter or a protein containing a peptide) upon elimination of the group R) to a carrier protein by a method known per se. A compound represented by the formula above can also be synthesized chemically by forming or introducing a carboxyl group, an amino group, or a sulfhydryl group in an appropriate starting material by a method known per se.

For example, a compound represented by the above-described formula wherein R is COOH and A is a polyoxyethylene alkylphenyl ether can be produced by dehydration condensation (half-esterifying) a polyoxyalkylphenyl ether and succinic anhydride (Cancer Biochemistry and Biophysics, 7. 175 (1984)). A compound represented by the above-described formula wherein R is NH₂ and A is a polyoxyethylene alkylphenyl ether can be produced by chlorinating the hydroxyl group of a polyoxyalkylphenyl ether with thionyl chloride (Journal of the American Chemical Society (J. Am. Chem. Soc.). 60. 2540 (1938)), followed by ammonia treatment (Organic Functional Group Preparations, Vol. 1, page 382). A compound represented by the above-described formula wherein R is SH and A is a polyoxyethylene alkylphenyl ether can be produced by chlorinating the hydroxyl group of a polyoxyalkylphenyl ether with thionyl chloride (Journal of the American Chemical Society (J. Am. Chem. Soc.). 60. 2540 (1938)), followed by a reaction with sodium hydrosulfide (Journal of the American Chemical Society (J. Am. Chem. Soc.). 72. 1843 (1950)).

As the above-described carrier protein, a variety of carrier proteins that have traditionally been used widely in the art, for example, KLH (keyhole limpet hemocyanin), BSA (bovine serum albumin), OVA (ovalbumin) and the like, can be mentioned.

The present invention also provides a monoclonal antibody produced by the antibody-producing hybridoma described above, and a method of monoclonal antibody production comprising a step using the above-described hybridoma. The monoclonal antibody of the present invention can be used as a reagent for quantitative determination of a desired antigenic substance (hereinafter also referred to as desired substance), and used for production of an affinity column for concentrating the above-described desired antigenic substance wherein the monoclonal antibody is immobilized to various carriers.

As examples of immobilization carriers for the monoclonal antibody of the present invention, microplates (e.g., 96-well microplates, 24-well microplates, 192-well microplates, 384-well microplates and the like), test tubes (e.g., glass test tubes, plastic test tubes), glass particles, polystyrene particles, modified polystyrene particles, polyvinyl particles, latexes (e.g., polystyrene latex), nitrocellulose membranes, cyanogen-bromide-activated filter paper, DBM-activated filter paper, particulate solid phases (e.g., Sepharose, Sephadex, agarose, cellulose, Sephacryl and the like), iron-containing polycarbonate membranes, magnet-containing beads and the like can be mentioned. The monoclonal antibody obtained using the present invention can be carried on carriers by the method described in “Enzyme Immunoassay”, pages 268 to 296, “Affinity Chromatography Handbook”, (Amersham-Pharmacia Biotech K.K., published Dec. 20, 1998)), which is a method known per se.

The present invention also provides a method of screening for an antigenic substance. The screening method of the present invention comprises a step using a monoclonal antibody produced by an antibody-producing hybridoma prepared by fusing a B cell, previously immunized with an antigenic substance in the coexistence of a cytokine and a glycolipid in vitro, and a myeloma cell. Although an appropriate conventionally known substance or a substance that will be newly obtained in the future can be used as the desired substance without limitation, the above-described endocrine disrupter or a protein containing a peptide, is preferred.

The screening method of the present invention is specifically performed by screening for an antigenic substance (desired substance) using a monoclonal antibody produced by a hybridoma prepared through the above-described step (the method of hybridoma production of the present invention). As methods of measuring the above-described desired substance, various methods in common use for detection of antigenic substances, such as radioisotope immunoassay (RIA method), ELISA method (Engvall, E., Methods in Enzymol., 70, 419-439 (1980)), fluorescent antibody method, plaque method, spot method, agglutination method, and Ouchterlony method (“Hybridoma Method and Monoclonal Antibodies”, published by R&D Planning, pages 30-53, Mar. 5, 1982) can be mentioned. From the viewpoint of sensitivity, ease of operation and the like, the ELISA method is used commonly.

The monoclonal antibody of the present invention can also be used in immunological concentration methods. Specifically, the desired substance of minimal immunological impurity contents can be concentrated at as high rates as several thousands to several tens of thousands of times, by passing a large amount of sample through an immunological adsorbent column or mixing the sample with immunological adsorbent particles to allow the above-described desired substance to be captured by the immunoadsorbent using an antigen-antibody reaction, and subsequently eluting the desired product by a known method, such as changing the pH (lowering the pH to 2.5 to 3, raising the pH to 11.5, and the like), changing the ionic strength (1M NaCl and the like), changing the polarity (10% dioxane, 50% ethylene glycol, 3M chaotropic salts (SCN⁻, CCl₃COO⁻, I⁻) and the like), adding a protein denaturant (8M urea, 6M guanidine hydrochloride and the like), or dissociating the desired substance by electrophoresis. In cases where the above-described desired substance is an endocrine disruptor, the endocrine disruptor, which occurs in very trace amounts in the environment, degradation products thereof, or a mixture thereof, can thereby be concentrated at much higher rates than by conventional methods of concentration such as solvent extraction and solid phase extraction, and in addition concentrates of lower contents of impurities and the like that interfere with quantitation can be obtained.

The present invention also provides a reagent containing at least a cytokine and a glycolipid. Preferable cytokines and glycolipids are as described above, with preference given to a combination of interleukin-4 as the cytokine and a lipopolysaccharide as the glycolipid. The reagent is useful for cell fusion, especially for cell fusion of an in vitro immunized B cell and a myeloma cell, and makes it possible to advantageously embody the method of antibody-producing hybridoma production and method of monoclonal antibody production of the present invention when used in combination with an appropriate B cell and myeloma cell.

### EXAMPLES

The present invention is hereinafter described in more detail by means of the following examples, which, however, are not to be construed as limiting the present invention.

### Example 1

[1] Cultivation of Myeloma Cells

(1) Thawing of Myeloma Cells

Myeloma cells of the BALB/c mouse-derived PAI line (Stocker et al., 1982) were used. Preserved PAI cells in liquid nitrogen were quickly thawed at 37° C. and transferred to a clean bench, and the following aseptic operation was performed.

First, the cells were transferred to a centrifugal tube containing 10 ml of previously prepared RPMI1640 complete medium (90% RPMI1640 (Roswell Park Memorial Institute 1640) (manufactured by Nissui Pharmaceutical)+10% FCS (fetal calf serum) (manufactured by Dainippon Pharmaceutical)+100 μg/ml kanamycin sulfate (manufactured by Meiji Seika Kaisha, Ltd.)+2 mM L-glutamine (manufactured by Nissui Pharmaceutical)+50 μM β-mercaptoethanol), and mixed gently.

Subsequently, the mixture was centrifuged at room temperature and 800 rpm (130×g) for 5 minutes; the resulting supernatant was removed, and the cell precipitate was suspended in 2.5 ml of RPMI1640 complete medium. Finally, the suspension was transferred to a T-25 culture flask and cultured at 37° C. in a 5% carbon dioxide incubator (manufactured by SANYO Electric Co., Ltd.). Next day, an additional 2.5 ml of RPMI1640 complete medium was added to the flask, and the suspension was subcultured every 2 to 3 days.

(2) Subculture of Myeloma Cells

Ordinary subculture was performed with myeloma cells adhering to the bottom of the T-25 culture flask detached by three to four gentle patting actions, and diluted 5 to 10 fold with RPMI1640 complete medium. Because cell fusion requires a large number of myeloma cells, a scaleup was started 1 week before cell fusion.

First, myeloma cells were counted and cultured at a density of 1×10⁵ cells/ml using a T-75 culture flask. Two days before cell fusion, myeloma cells in dilution at a density of 1×10⁵ cells/ml were cultured with a scaleup to three T-150 culture flasks.

[2] Preparation of Antigenic Substance

Di-2-ethylhexyl phthalate (DEHP) is of low antigenicity because it is a low-molecular antigenic substance. Hence, a DEHP derivative was prepared by the method described below.

Preparation of DEHP Derivative Hapten):

After 10 g of 8-bromooctanoic acid was dissolved in 300 ml of tetrahydrofuran (THF), 20 ml of diphenylaminomethane was added, and the reaction was carried out at room temperature overnight. Next day, 20 ml of diphenylaminomethane was added, and the reaction was carried out at room temperature overnight. After concentration under reduced pressure, the reaction product was dissolved in hexane-ethyl acetate (9:1) and crudely purified using a silica gel column.

20 g of this crudely purified substance, 2.42 g of phthalic acid, and 2.22 g of 1,8-diazabicycloundecene (DBU) were refluxed under heating in 60 ml of benzene overnight. Next day, 2.22 g of DBU was added, the ingredients were refluxed under heating for 6 hours and cooled to room temperature, water and chloroform were added, and the mixture was washed with water. After dehydration and concentration, the chloroform layer was dissolved in hexane-ethyl acetate (9:1) and crudely purified using a silica gel column.

4.1 g of this crudely purified substance was dissolved in 100 ml of THF, and 0.4 g of 10% Pd/C (50% hydrated) was added. After H₂ sparging (0.3 ml/minute, 5 hours), an additional 1.2 g of 10% Pd/C was added. After further H₂ sparging (0.5 ml/minute, 2 hours), the catalyst was removed, and the solution was concentrated under reduced pressure. After the resulting concentrate was dissolved in 75% methanol, the resulting solution was purified using an ODS column to yield 1.9 g of the desired substance (DEHP derivative).

To increase the antigenicity, KLH (keyhole limpet hemocyanin) was used as the carrier protein and DEHP derivative-KLH, which is a DEHP derivative-carrier protein complex with KLH previously bound thereto, was used as the antigenic substance.

[3] Preparation of Mouse Splenocytes

(1) Acquisition of Serum for Antibody Titer Determination

BALB/c mice at 4 to 10 weeks of age (SPF breed, female) were used. Each mouse was given an intraperitoneal injection of 50 μl of 0.1 g/ml chloral hydrate; after the mouse became inactive, an additional 50 μl was injected. After confirmation of complete anesthesia, the mouse was immobilized onto an anatomical stage using injection needles. After the mouse abdomen was disinfected with 70% alcohol, an incision was made using anatomical scissors, and the incision was expanded nearly to the heart. Subsequently, the animal was laparotomized to expose the heart with endothelium close to the heart picked with tweezers, and blood was quickly drawn from the heart. The blood sample obtained was clotted, then centrifuged at 4° C. and 6700×g for 5 minutes to separate serum. The antibody titer of the serum obtained was determined by the ELISA method described below.

(2) Preparation of Splenocytes

After heart blood drawing, the mouse was sterilized by immersion in a 300-ml beaker containing 70% ethanol, placed in a clean bench, and anatomized to extirpate splenocytes.

First, the outer skin of the mouse was raised with tweezers, an incision was made in the left flank using anatomical scissors, and endothelium was cut to expose the spleen. Subsequently, the spleen was drawn out from the body using tweezers, the fat around the spleen was removed using scissors, and the spleen was extirpated from the mouse. The extirpated spleen was washed several times in a petri dish containing 10 ml of RPMI1640 containing an antibiotic (100 μg/ml kanamycin), and the peripheral fat was removed using scissors. The spleen was placed on a stainless steel mesh immersed in another petri dish, and gently milled with a rubber policeman.

Subsequently, the spleen suspension was transferred to a 50-ml centrifugal tube. The stainless steel mesh was washed with 10 ml of the antibiotic-containing RPMI1640, and the suspension was added to a 50 ml tube. This operation was repeated until the liquid volume became 40 ml. Subsequently, the suspension was centrifuged at 2000 rpm (800×g) for 5 minutes to yield prepared splenocytes.

[4] In Vitro Immunization of Mouse Splenocytes

The splenocyte precipitate prepared in [3] (2) above was suspended in 5 ml of an antibiotic-containing RPMI1640, and the resulting suspension was dispensed to T-25 culture flasks at 2.5 ml/flask. 100 μl of 1 mg/ml adjuvant peptide (N-acetylmuramyl-L-alanyl-D-isoglutamine) (manufactured by SIGMA) was added to each flask, and the contents were mixed by gently shaking the flask. Furthermore, 20 μl or 5 μl of the antigen solution of DEHP derivative-KLH (1 mg/ml) was added to each flask, and the flask was allowed to stand for 15 minutes. Finally, 2.5 ml of a previously prepared complete medium containing 40% FCS (60% RPMI1640+100 μg/ml kanamycin sulfate+2 mM L-glutamine+50 μM β-mercaptoethanol+40% FCS) was added to each flask. Furthermore, 20 μl (final concentration: 10 ng/ml) of 5 μg/ml interleukin-4 (11020, manufactured by SIGMA) and 200 μl (final concentration: 20 μg/ml) of 1 mg/ml lipopolysaccharide (L4391, manufactured by SIGMA) were added, and the contents were gently mixed and cultured in a 5% carbon dioxide incubator for 4 days.

After being allowed to stand in the 5% carbon dioxide incubator for 4 days, immunized mouse splenocytes were centrifuged at 800×g for 5 minutes, then suspended in 10 ml of an antibiotic-containing RPMI1640, again centrifuged at 800×g for 5 minutes, and washed. After centrifugation, the resulting precipitate was suspended in 2.5 ml of the antibiotic-containing RPMI1640 to yield a suspension containing splenocytes immunized in vitro.

[5] Cell Fusion by the PEF (Pulsed Electric Field) Method and Visualized Analysis

(1) Preparation of Immunized Cell-Antigenic Substance (DEHP Derivative)-Avidin Complex

20 μl (1 mg/ml) of DEHP derivative-Av (antigenic substance-avidin conjugate) was added to 2.5 ml of a kanamycin-containing RPMI1640, and 2.5 ml of a suspension of splenocytes immunized in vitro by the above-described method was added. After rotation at 4° C. for 2 hours, centrifugation was conducted at 800×g for 5 minutes, and the resulting precipitate was washed with 10 ml of the kanamycin-containing RPMI1640. Finally, the precipitate was suspended in 5 ml of the kanamycin-containing RPMI1640.

(2) Preparation of Biotin-Myeloma Cell Complex

Myeloma cells cultured with a scaleup were recovered and centrifuged at 130×g for 5 minutes, and the resulting precipitate was suspended in 40 ml of PBS (phosphate-buffered saline). After the resulting suspension was again centrifuged at 130×g for 5 minutes, the resulting precipitate was suspended in 5 ml of PBS. Separately, 30 μl of N-hydroxysuccinimide-biotin (NHS-biotin) (1 mg/30 μl in DMF) was added to 5 ml of PBS, and the two suspensions were quickly mixed and gradually rotated at 37° C. in a 5% carbon dioxide incubator for 30 minutes. Subsequently, centrifugation was conducted at 130×g for 5 minutes, and the resulting cell precipitate was washed with 50 ml of a kanamycin-containing RPMI1640. The biotinized myeloma cells were suspended in 5 ml of the antibiotic-containing RPMI1640.

(3) Preparation and Electric Fusion of Immunized Cell-Antigenic Substance (DEHP Derivative)-Avidin-Biotin-Myeloma Cell Complex

Each suspension prepared in. [5] (1) and (2) above was mixed so that the ratio of the splenocyte-antigenic substance-avidin complex and the myeloma cell-biotin complex would be 1:1. This suspension was centrifuged at 1000 rpm (200×g) for 10 minutes, and the resulting precipitate was suspended in 1 ml of a kanamycin-containing RPMI1640. After further centrifugation at 500 rpm (50×g) for 1 to 2 minutes, the suspension was allowed to stand in a clean bench for 30 minutes. Subsequently, the suspension was slowly rotated in a 5% carbon dioxide incubator for 30 minutes. After rotation, the suspension was centrifuged at 200×g for 10 minutes, and the resulting precipitate was suspended in 2 ml of an isotonic sucrose buffer (0.25 M sucrose+2 mM sodium dihydrogen phosphate/disodium hydrogen phosphate (pH 7.2)+0.1 mM magnesium chloride+0.1 mM calcium chloride). This suspension was added to platinum plates of the preparation type at 0.5-ml to 1.0-ml per plate, and electric fusion was conducted using a cell fusion apparatus (electro square porator T820, manufactured by BTX) under the conditions of 2 kV/cm (10 μsec×4 times) and 3 kV/cm (10 μsec×4 times).

After electric fusion, the fused cell suspension was gently added to 20 ml of previously prepared RPMI1640 complete medium, and the suspension was allowed to stand for 30 minutes and dispensed to a 96-well plate at 0.2 ml/well. Subsequently, the plate was quickly transferred to a 5% carbon dioxide incubator and cultured at 37° C. Next day, the culture supernatant was removed from the plate at 0.1 ml/well, and HAT (100 μM hypoxanthine+0.4 μM aminopterin+16 μM thymidine) (manufactured by SIGMA), previously diluted 50 fold with RPMI1640 complete medium, was added at 0.1 ml/well.

(4) Visualized Analysis of Immunized Cells (Preparation of Immunized Cell-Antigenic Substance-Avidin-Biotin-Phycoerythrin Complex)

Regarding the selection of immunized cells, the most important step in the PEF method, visualized analysis was conducted using a specific fluorescent label per the procedure shown below.

Splenocytes immunized in vitro were suspended in 2.5 ml of RPMI1640, and the resulting suspension was mixed with 2.5 ml of RPMI1640 containing 20 μl of DEHP derivative-avidin conjugate and rotated at 4° C. for 2 hours. Subsequently, the suspension was centrifuged at 800×g for 5 minutes, the resulting precipitate was suspended in 10 ml of Hanks balanced salt solution, the resulting suspension was again centrifuged at 800×g for 5 minutes and was washed. The precipitate was suspended in 0.5 ml of Hanks balanced salt solution, 20 μl of biotin-phycoerythrin (biotin-PE) (manufactured by Biomeda Corporation) was added, and the suspension was rotated at room temperature under shading conditions for 1 hour. After completion of rotation, the suspension was centrifuged at 800×g for 5 minutes, the resulting precipitate was suspended in 10 ml of Hanks balanced salt solution, the resulting suspension was again centrifuged at 800×g for 5 minutes and was washed. Finally, the precipitate was suspended in 0.5 ml of Hanks balanced salt solution and examined using a confocal laser microscope.

As a result, some cells showing specific fluorescence were identified. From this finding, it was considered that the desired immunized cell was recognized specifically by the DEHP derivative and formed an immunized cell-antigenic substance-avidin complex. Hence, the in vitro immunization method was demonstrated to be also effective on endocrine disruptors that are low-molecular functional antigenic substances.

When using a splenocyte suspension not sensitized with an antigenic substance, no specifically fluorescently labeled cells were observed even with the addition of DEHP derivative-avidin and biotin-PE.

[6] Screening for and Cloning of Monoclonal Antibody-Producing Hybridoma

(1) ELISA (Enzyme-Linked Immunosorbent Assay) Method

Individual wells were examined for hybridoma colonization using an inverted phase contrast microscope. For each well with a sign of colonization, the culture supernatant was collected and examined for the desired antibody-producing hybridoma by the ELISA method per the procedure shown below.

DEHP derivative-OVA (DEHP derivative-ovalbumin), as the antigenic substance, was diluted with PBS (pH 7.2) to a concentration of 10 μg/ml, and the resulting dilution was added to a 96-well plate at 50 μl/well and allowed to stand at 4° C. for one day to adsorb the antigenic substance to the plate. Subsequently, after the plate was twice washed with PBS, PBS-diluted 3% gelatin was added at 350 μl/well, and the plate was allowed to stand at 4° C. for one day to achieve blocking. The plate was further allowed to stand at 37° C. for 1 hour, then washed with PBST (phosphate-buffered saline in 0.05% Triton X-100) three times, a primary antibody as the culture supernatant was added at 50 μl/well, and the plate was incubated at 37° C. for 1 hour. Subsequently, after the plate was washed with PBST three times, a secondary antibody (goat anti-mouse IgG(H+ L) conjugated with HRP (horseradish peroxidase) (manufactured by Biosource International, Inc.)), previously diluted with PBST 10000 fold, was added at 50 μl/well, and the plate was incubated at 37° C. for 1 hour. Finally, after the plate was washed with five times, a color developer (0.1M sodium citrate buffer (pH 5.2)+o-phenylene diamine (1 mg/ml)+0.02% H₂O₂) was added at 100 μl/well, the plate was incubated at 37° C. for 15 minutes to develop a color, and 1M sulfuric acid was added at 50 μl/well to stop the reaction. The OD₄₉₀ₙₘ of this plate was measured using a microplate reader (manufactured by Bio-Rad Laboratories).

(2) HAT Selection and HT Selection

The culture supernatant in the 96-well plate was exchanged with a HAT-containing RPMI complete medium (100 μM hypoxanthine+0.4 μM aminopterin+16 μM thymidine) (manufactured by SIGMA) at 0.1 ml/well every 2 to 3 days for two weeks. After two weeks elapsed, the HAT-containing RPMI medium was replaced with a HT-containing RPMI complete medium (100 μM hypoxanthine+16 μM thymidine) (manufactured by SIGMA), cultivation with medium exchanges was conducted in the same manner for 2 weeks, and this was followed by cultivation with medium exchanges with RPMI1640 complete medium.

(3) Limiting Dilution

Desired antibody-producing hybridomas that tested positive in the ELISA method in [6] (1) above (ELISA-positive wells) were carefully detached from the wells using a Pasteur pipette, and cells were counted. Subsequently, the cells were diluted with RPMI1640 complete medium to obtain cell densities of 9 cells/well, 3 cells/well, 1 cell/well, and 0.5 cells/well, and each dilution was dispensed to 12 wells of a 96-well plate at 0.2 ml/well, respectively. Subsequently, the plate was cultured at 37° C. in a 5% carbon dioxide incubator. After subculture for 2 weeks, and before medium exchanges, each well was examined for formation of hybridomas using a phase contrast microscope. In cases where hybridomas were found in 30% to 40% of the wells, the culture supernatant was collected and assayed for antibody titer by the ELISA method to determine the presence or absence of the desired antibody-producing hybridoma.

[7] Mass Culture and Preservation of Monoclonal Antibody-Producing Hybridoma

(1) Mass Culture of Hybridoma

After limiting dilution was once or twice repeated, a hybridoma exhibiting a high antibody titer as determined by the ELISA method (ΔOD>0.5) was cultured with a scaleup to a 24-well plate. Subsequently, the cultivation scale was increased to a T-25 culture flask and to a T-75 culture flask in this order. At each stage, the culture supernatant was collected and centrifuged at 800×g for 5 minutes, and the resulting supernatant was assayed for antibody titer by the ELISA method.

(2) Preservation of Hybridoma

The hybridoma cultured with a scaleup in [7] (1) above was centrifuged at 130×g for 5 minutes and suspended in 1.5 ml of 100% FCS. Subsequently, an equal amount (1.5 ml) of freezing buffer (80% RPMI1640 (manufactured by Nissui Pharmaceutical Co., Ltd.)+20% DMSO (manufactured by SIGMA)+4 mM L-glutamine (manufactured by Nissui Pharmaceutical Co., Ltd.)) was added to the hybridoma suspension in FCS, and mixed. This suspension was preserved at −80° C. in a freezer for 1 to 2 weeks. Subsequently, the suspension was transferred in dry ice to a liquid nitrogen preservation container.

The hybridoma culture supernatant was preserved in the presence of 0.05% NaN₃ at 4° C.

[8] Production of Monoclonal Antibody in Mouse Ascites Fluid

The hybridoma obtained in [7] (1) above was centrifuged at 130×g for 5 minutes, and the resulting precipitate was suspended in 0.5 ml of PBS. Cells were counted, and a cell density of not less than 1×10⁸ cells/ml was obtained. The hybridoma was injected to one mouse that had received an intraperitoneal injection of 0.5 ml of pristane (2,6,10,14-tetramethylpentadecane) 3 to 7 days previously. About 2 weeks later, when the mouse's lower abdomen swelled to the extent of gait difficulty, the mouse was killed by cervical dislocation, and the ascites fluid that had accumulated in the peritoneum was recovered. The peritoneal cavity was washed with PBS, and the washings were recovered. The ascites fluid obtained was centrifuged at 800×g for 5 minutes, the same centrifugal operation was twice repeated, and the resulting supernatant was preserved in the presence of 0.05% NaN₃ at 4° C.

[9] Purification of Monoclonal Antibody

Monoclonal antibody was purified from the mouse ascites fluid obtained in [8] above, using a protein G column. For column pretreatment, 5 ml of ultrapure water was injected into the column at a rate of one drop/sec using a syringe to wash down the ethanol for preservation. Subsequently, the inside of the column was equilibrated with a binding buffer, the mouse ascites fluid was mixed with an equal volume of the binding buffer, and the mixture was applied to the column. After the inside of the column was washed with the binding buffer until the OD₂₈₀ₙₘ of the column effluent became nearly zero, the monoclonal antibody was separated using the elution buffer. The OD₂₈₀ₙₘ of each fraction was determined, the antibody titer was determined by the ELISA method, and a fraction containing the desired monoclonal antibody was preserved in the presence of 0.05% NaN₃ at 4° C.

### Example 2

A hybridoma was prepared, and the desired monoclonal antibody was obtained, in the same manner as Example 1, except that human insulin (I0259, manufactured by SIGMA: total amount 25 μg) was used as the antigenic substance, biotin-B-1-II (total amount 20 μg: synthesized using an ordinary peptide synthesizer) or biotin-B-3-II (total amount 20 μg: synthesized sing an ordinary peptide synthesizer) was used as the B cell selection conjugate, and a complex with biotin-myeloma cell conjugate was subjected to cell fusion via streptavidin.

B-1-II and B-3-II used in the B cell selection conjugate correspond to a part of the amino acid sequence of the human insulin B chain, specifically to the amino acid sequences shown below.

(Sequence Listing SEQ ID NO: 1)

B-1-II: Val-Asn-Gln-His-Leu-Cys-Gly

(Sequence Listing SEQ ID NO: 2)

B-3-II: Phe-Phe-Tyr-Thr-Pro-Lys-Thr

### Comparative Example 1

A hybridoma was prepared, and the desired monoclonal antibody was obtained, in the same manner as Example 1, except that splenocytes were immunized in vitro in the absence of interleukin-4 and lipopolysaccharide.

### Comparative Example 2

A hybridoma was prepared, and the desired monoclonal antibody was obtained, in the same manner as Example 2, except that splenocytes were immunized in vitro in the absence of interleukin-4 and lipopolysaccharide.

Evaluation Result 1:

Hybridoma Positivity Rate and ELISA Positivity Rate

From the results of an examination for the presence or absence of hybridoma colonization using an inverted phase contrast microscope after conduct of the PEF method, the hybridoma positivity rate (=(number of hybridoma-positive wells/number of all wells)×100 (%)) was calculated.

The positivity rate of hybridoma-positive wells in ELISA (ELISA in Example 1 [6] (1)) (=(number of ELISA-positive wells/number of hybridoma-positive wells)×100 (%)) was also calculated.

The results of Example 1 are shown in Table 1; the results of Comparative Example 1 are shown in Table 2: the results of Example 2 are shown in Table 3; the results of Comparative Example 2 are shown in Table 4.

TABLE 1

| | Sample number | | | |
|---|---|---|---|---|
| | 1 | 2 | 3 | 4 |
| Applied voltage (kV/cm) | 2.0 | 3.0 | 2.0 | 3.0 |
| for PEF method | | | | |
| Total number of wells | 96 | 96 | 96 | 96 |
| Number of hybridoma-positive | 51 | 41 | 55 | 43 |
| wells | | | | |
| Number of ELISA-positive wells | 9 | 22 | 15 | 12 |
| Hybridoma positivity rate (%) | 53.1 | 42.7 | 57.3 | 44.8 |
| ELISA positivity rate (%) | 17.6 | 53.7 | 27.3 | 27.9 |

TABLE 2

| | Sample number | | |
|---|---|---|---|
| | 1 | 2 | 3 |
| Applied voltage (kV/cm) | 2.0 | 3.0 | 3.0 |
| for PEF method | | | |
| Total number of wells | 96 | 96 | 96 |
| Number of hybridoma-positive | 12 | 5 | 19 |
| wells | | | |
| Number of ELISA-positive wells | 4 | 1 | 5 |
| Hybridoma positivity rate (%) | 12.5 | 5.2 | 19.8 |
| ELISA positivity rate (%) | 33.3 | 20.0 | 26.3 |

TABLE 3

| | Sample number | | | |
|---|---|---|---|---|
| | 1 | 2 | 3 | 4 |
| Selection antigen | B-1-II | B-1-II | B-3-II | B-3-II |
| Applied voltage (kV/cm) | 2.0 | 3.0 | 2.0 | 3.0 |
| for PEF method | | | | |
| Total number of wells | 48 | 48 | 48 | 48 |
| Number of hybridoma- | 38 | 41 | 24 | 40 |
| positive wells | | | | |
| Number of ELISA-positive | 3 | 6 | 1 | 6 |
| wells | | | | |
| Hybridoma positivity rate (%) | 79.2 | 85.4 | 50.0 | 83.3 |
| ELISA positivity rate (%) | 7.9 | 14.6 | 4.2 | 15.0 |

TABLE 4

| | Sample number | | | |
|---|---|---|---|---|
| | 1 | 2 | 3 | 4 |
| Selection antigen | B-1-II | B-1-II | B-3-II | B-3-II |
| Applied voltage (kV/cm) | 2.0 | 3.0 | 2.0 | 3.0 |
| for PEF method | | | | |
| Total number of wells | 48 | 48 | 48 | 48 |
| Number of hybridoma- | 2 | 1 | 1 | 1 |
| positive wells | | | | |
| Number of ELISA-positive | 0 | 0 | 0 | 0 |
| wells | | | | |
| Hybridoma positivity rate (%) | 4.2 | 2.1 | 2.1 | 2.1 |
| ELISA positivity rate (%) | 0 | 0 | 0 | 0 |

From the results above, it is evident that the hybridomas of Examples 1 and 2, which resulted from cell fusion by the PEF method following a step of in vitro immunization of a B-cell in the coexistence of interleukin-4 and a lipopolysaccharide, emerge at a much higher ratio, compared to the hybridomas of Comparative Examples 1 and 2, which were obtained without the same step.

Evaluation Result 2:

Antibody Class Typing

For samples of the hybridomas prepared and cloned in Examples 1 and 2 and Comparative Examples 1 and 2 (each well showing a measured value of not less than 0.5 as determined by the ELISA method is assumed to constitute one sample), the class of the monoclonal antibody produced by each hybridoma was determined by the sandwich ELISA method (Tandem method). The sandwich ELISA method was performed per the procedure shown below.

Each of anti-mouse IgM antibody, anti-mouse IgG₁ antibody, anti-mouse IgG₂ₐ antibody, anti-mouse IgG_{2b} antibody, and anti-mouse IgG₃ antibody (all manufactured by Cosmo Bio Co., Ltd.) was diluted with 0.1 M NaHCO₃ (pH 9.8) to obtain a concentration of 10 μg/ml, each dilution was added to a 96-well plate at 50 μl/well, and the plate was allowed to stand at 4° C. for one day to adsorb each antigenic substance (anti-mouse antibody) to the plate. Subsequently, the plate was washed with PBS three times, 1% gelatin was added at 350 μl/well, and the plate was incubated at 37° C. for 1 to 2 hours to achieve blocking. After the plate was washed with PBST three times, the cell culture supernatant was added at 25 μl/well, and the plate was incubated at 37° C. for 1 hour. After the plate was washed with PBST three times, a secondary antibody, previously diluted with PBST 5000 fold, was added by 50 μl/well, respectively, and the plate was incubated at 37° C. for 1 hour. Finally, after the plate was washed with PBST five times, a color developer (the same as [6] (1) above) was added at 50 μl/well, the plate was incubated at 37° C. for 15 minutes to develop a color, and 1 M H₂SO₄ was added at 50 μl/well to stop the reaction. The OD₄₉₀ₙₘ of this plate was measured using a microplate reader to determine the type of the monoclonal antibody.

The results of Example 1 and Comparative Example 1 are shown in Table 5.

TABLE 5

| | Sample No. | IgM type | IgG type |
|---|---|---|---|
| Example 1 | 1 | 0.34 ± 0.08 | 0.70 ± 0.16 |
| | 2 | 0.37 ± 0.19 | 0.56 ± 0.18 |
| | 3 | 0.22 ± 0.08 | 0.53 ± 0.16 |
| | 4 | 0.22 ± 0.08 | 0.58 ± 0.16 |
| Comparative | 1 | 0.55 ± 0.44 | 0.12 ± 0.02 |
| Example 1 | 2 | 0.57 ± 0.02 | 0.14 ± 0.01 |
| | 3 | 0.58 ± 0.04 | 0.37 ± 0.04 |
| | 4 | 0.40 ± 0.24 | 0.08 ± 0.04 |
| | 5 | 0.55 ± 0.25 | 0.21 ± 0.05 |

From the results above, it is evident that the monoclonal antibodies of Examples 1 and 2, which were obtained via a step of in vitro immunization of a B cell in the coexistence of interleukin-4 and a lipopolysaccharide, are of the IgG type and produced at a much higher ratio, compared to the monoclonal antibodies of Comparative Examples 1 and 2, which were obtained without the same step.

Evaluation Result 3:

Effects on In Vitro Immunization

As described above, results showing that the desired immunized cell was specifically recognized by the DEHP derivative-avidin conjugate were obtained from the visualized analysis of splenocytes using a specific fluorescent label in Example 1 [5] (4).

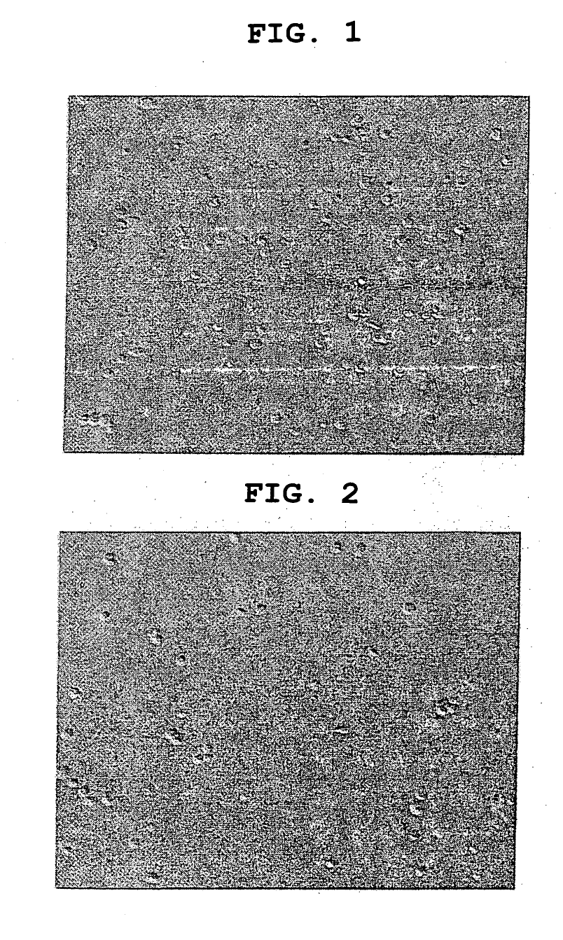

FIG. 1 is a photomicrograph showing the results of the visualized analysis in Example 1, and FIG. 2 is a photomicrograph showing the results of the visualized analysis in Comparative Example 1 (both taken using a confocal laser microscope (magnified 300 fold)). The above-described visualized analyses also demonstrated that the number of specifically recognized splenocytes increased remarkably in the coexistence of interleukin-4 and a lipopolysaccharide during the above-described in vitro immunization, compared to the absence of interleukin-4 and the lipopolysaccharide.

During the above-described visualized analyses, cells were counted using a hemocytometer prior to observation under a confocal laser microscope; during 4 days of in vitro immunization, the number of splenocytes as a whole increased nearly 10 fold (Example 1: 4.7×10⁷ cells to 5.4×10⁷ cells, Comparative Example 1: 0.58×10⁷ cells); this result suggests cell proliferation in the coexistence of interleukin-4 and a lipopolysaccharide during in vitro immunization.

### INDUSTRIAL APPLICABILITY

As is evident from the description above, the hybridoma obtained using the production method of the present invention emerges at a much higher ratio (hybridoma positivity rate) compared to the conventional method. Furthermore, the hybridoma obtained using the production method of the present invention is capable of producing a monoclonal antibody of the IgG type, which is a monomer and is structurally stable, at a much higher ratio than the IgM type, which is a pentamer and is structurally unstable. Using this method of hybridoma production, it is possible to provide a method of monoclonal antibody production, cell fusion reagent, antigenic substance screening method and the like that are much more useful than conventional counterparts.

1-18. (canceled)

19. A method of producing an antibody-producing hybridoma, which comprises fusing a B cell, previously immunized with an antigenic substance in the coexistence of a cytokine at a sufficient concentration to selectively produce an IgG type antibody, and a glycolipid in vitro, and a myeloma cell.

20. The method of claim 19, wherein the cytokine concentration ranges from about 1 ng/ml to about 100 ng/ml.

21. The method of claim 19, wherein the cytokine concentration ranges from about 5 ng/ml to about 30 ng/ml.

22. The method of claim 19, wherein a cell is immunized in the absence of Phytolacca americana lectin and/or killed Staphylococcus aureus cells coated with protein A.

23. The method of claim 20, wherein a cell is immunized in the absence of Phytolacca americana lectin and/or killed Staphylococcus aureus cells coated with protein A.

24. The method of claim 21, wherein a cell is immunized in the absence of Phytolacca americana lectin and/or killed Staphylococcus aureus cells coated with protein A.

25. The method of claim 19, wherein the cytokine is interleukin-4 and the glycolipid is lipopolysaccharide.

26. The method of claim 19, wherein the antigenic substance is an endocrine disruptor or a protein containing a peptide.

27. The method of claim 19, which comprises cell fusion by the pulsed electric field method.

28. The method of claim 26, which comprises cell fusion using (a) and (b) below:
(a) an immunized cell joined to a complex comprising both the antigen used for immunization and one member of a pair of specific couplings, and
(b) a myeloma cell joined to the other member of the pair of specific couplings.

29. A monoclonal antibody produced by a hybridoma prepared by the method of claim 19.

30. A cell fusion reagent containing at least both a cytokine at a sufficient concentration to selectively produce an IgG type antibody, and a glycolipid.

31. The cell fusion reagent of claim 30, wherein the cytokine is interleukin-4, and the glycolipid is lipopolysaccharide.

32. A method of monoclonal antibody production, which comprises a step using an antibody-producing hybridoma obtained by fusing a B cell, previously immunized with an antigenic substance in the coexistence of a cytokine at a sufficient concentration to selectively produce an IgG type antibody, and a glycolipid in vitro, and a myeloma cell.

33. The production method of claim 32, wherein the antigenic substance is an endocrine disruptor or a protein containing a peptide.

34. A method of screening for an antigenic substance, which comprises a step using a monoclonal antibody produced by an antibody-producing hybridoma obtained by fusing a B cell, previously immunized with an antigenic substance in the coexistence of a cytokine at a sufficient concentration to selectively produce an IgG type antibody, and a glycolipid in vitro, and a myeloma cell.

35. The screening method of claim 34, herein the antigenic substance is an endocrine disruptor or a protein containing a peptide.

36. A method of hybridoma production, which comprises cell fusion of (a) and (b) below by the pulsed electric field method:
(a) an immunized cell joined to a complex comprising both the antigen used for immunization and one member of a pair of specific couplings, and
(b) a myeloma cell joined to the other member of the pair of specific couplings.


SEQUENCE LISTING (2 sequences)

SEQ ID NO: 1 - protein, 7 aa, Artificial Sequence
  Partial amino acid sequence from human insulin B chain
VNQHLCG

SEQ ID NO: 2 - protein, 7 aa, Artificial Sequence
  Partial amino acid sequence from human insulin B chain
FFYTPKT